(19) United States
(12) Patent Application Publication
(10) Pub. No.: US 2002/0168327 A1
(43) Pub. Date: Nov. 14, 2002

(54) Treatment of dandruff

(75) Inventors: Peter Lawrence Bailey, Wirral (GB); Clive Roderick Harding, Bedford (GB); Christopher John Little, Wirral (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/084,269

(22) Filed: Feb. 26, 2002

(30) Foreign Application Priority Data
Feb. 27, 2001 (GB) 0104886.7
Apr. 20, 2001 (EP) 01303670.2

(51) Int. Cl. A61K 31/555; A61K 7/06

(52) U.S. Cl. 424/70.1; 514/184

(57) ABSTRACT

Dandruff and symptoms of dandruff can be treated using lipophilic agents or lipid precursor and/or promoters to strengthen the scalp. The lipophilic agents or lipid precursor and/or promoters are preferably used in conjunction with an antifungal agent which has activity against Malassezia spp, particularly Malassezia furfur. Suitable antifungal agents include those conventionally used for the treatment of dandruff including, for example, zinc pyrithrone (ZnPTO), octopirox and azole antifungal agents such as climbazole and ketocanazole. The antifungal agents may be used singularly or as a mixture of one or more antifungal agents.

### FIELD OF THE INVENTION

This invention relates to the treatment and/or prevention of dandruff and symptoms of dandruff. In particular, the invention relates to the use of a class of compounds for treating dandruff, for strengthening the scalp and for reducing scalp itch and/or dryness.

### BACKGROUND AND PRIOR ART

It is widely believed that Malassezia yeasts, such as Malassezia furfur, are the main cause of dandruff. However, it is unclear why some people suffer from this condition while others do not. What is known is that increasing the level of Malassezia on the scalp does not automatically lead to dandruff. This suggests that Malassezia is necessary but not sufficient to cause the condition.

The main, if not only, intervention strategy used on the market currently for the treatment of dandruff is the topical application of antifungals such as zinc pyrithione (ZnPTO), octopirox, climbazole and ketoconazole which are normally delivered from a shampoo. These antifungal agents remove (or at least reduce the level of) the Malassezia from the scalp, and provide moderately effective treatment of the dandruff condition.

However, despite this there is widespread dissatisfaction with the performance of current anti-dandruff shampoos from dandruff sufferers globally. Clinical studies have revealed that one reason behind this situation is that many people who believe they have dandruff do not actually exhibit the clinical symptoms of dandruff that anti-fungal based anti-dandruff shampoos are designed to treat. Many people who claim to suffer from dandruff present a visibly healthy looking scalp despite suffering from perceptible scalp dryness and itch. The definition of dandruff for the purposes of this invention therefore includes the total population of people who claim to suffer from dandruff and is not restricted to those people simply presenting recognised clinical symptoms of the condition.

Shampoo products are available that contain active agents for dry and itching scalp. For example, Eucerin (TM) from Beiersdorf contains urea as active agent as well as skin related lipids and other components.

It is known that lipophilic agents can be applied to the skin and/or to the hair.

For example, WO99/18919 describes the inclusion of certain oils, together with dehydroacetic acid, as an optional component in a shampoo. The teaching in this document generally relates, however, to topical compositions for preventing an oily/shiny appearance of the skin. The document claims that by reducing the production of sebum and hence making the skin less oily, the compositions are able to control a number of skin conditions.

DE-A-4419783 describes the use of a medicated shampoo containing lipoic acids or their derivatives which have a pharmacological effect. There is no teaching in this document of physically treating the scalp or of antifungal agents.

EP-A-0872229 describes the use of antifungal agents ketoconazole and selenium sulphide in conjunction with a synthetic amphoteric phospholipid delivered from a shampoo. This document teaches that the phospholipid works with the antifungal agent to give increased anti-fungal activity. Furthermore, it teaches that dandruff is often accompanied by high or excessive oil or sebum production.

U.S. Pat. No. 5,869,034 teaches that sphingolipids may be used to protect skin or hair from heavy metals present in atmospheric pollution.

The use of ceramides to prevent the loss of proteins from the skin or from hair is described in U.S. Pat. No. 5,939,077.

A two-part composition for the treatment of dandruff is described in WO88/00041. The treatment involves a final step of treating the hair with a solution at an acidic pH. The document claims that a protective effect is obtained by suppression of the production of sebum.

WO95/02389 discloses high lathering, anti-dandruff shampoo compositions containing a particulate anti-dandruff active and a crystalline suspending agent for the active. The anti-dandruff activity of the compositions is not described as being attributable to anything other than the particulate active.

U.S. Pat. No. 5,565,207 describes a scalp-moisturiser composition containing steroid glycoside and/or triterpenoid glycoside, sphingo glycolipid and steroid hormone, in defined amounts and ratios. The compositions are stated as suppressing dandruff formation through the physiological effects of the specific compositions which are claimed to normalise scalp physiology.

The use of alpha-hydroxy acids (AHAs) in the treatment of a range of skin conditions, including dandruff, is described in U.S. Pat. Nos. 3,984,566, 3,988,470, 4,105,782 and 4,363,815. However, none of these documents teaches the need to restore the scalp barrier function via the increase in lipid production in the scalp. The documents describe the skin conditions as being characterised by overproduction of keratinocytes and their retention in the stratum corneum for abnormally long periods. Therefore, the mode of action of the AHAs is primarily attributed to a reduction in the thickness of the hyperkeratotic stratum corneum by reducing corneocyte adhesion at lower levels of the stratum corneum.

The present invention aims to provide a system for treating dandruff which has advantages over the prior art methods and compositions. The invention is based on the surprising finding that treating the scalp in order to boost the level of lipid within the scalp, or with a lipophilic agent, rather than suppressing the production of sebaceous materials as taught by the prior art, can be effective in treating dandruff.

### SUMMARY OF THE INVENTION

According to the invention, there is provided the use of a lipophilic agent or a lipid precursor and/or promoter in the manufacture of a composition for treating and/or preventing dandruff by strengthening the scalp.

Also provided by the invention in another aspect is a method of treating and/or preventing dandruff which comprises strengthening the scalp by applying to the scalp a composition comprising a lipophilic agent or a lipid precursor and/or promoter in an amount effective to strengthen the scalp.

Further provided by the invention is the use of a lipophilic agent or a lipid precursor and/or promoter for strengthening the scalp.

Also provided by the invention is the use of a lipophilic agent or a lipid precursor and/or promoter for reducing scalp itch and/or dryness.

Another embodiment of the invention is a method of strengthening the scalp which comprises applying to the scalp a composition comprising a lipophilic agent or lipid precursor and/or promoter in an amount effective to strengthen the scalp.

A further embodiment of the invention is a method of reducing scalp itch and/or dryness which comprises strenghthening the scalp by a method according to the invention.

### DETAILED DESCRIPTION OF THE INVENTION

The present invention involves the treatment of the scalp with a lipophilic agent or a stratum corneum lipid precursor and/or promoter. Preferably, the lipid precursor and/or promoter is a precursor and/or promoter of a ceramide.

Ultrastructurally and biochemically, the stratum corneum of the scalp is very similar to that of the general interfollicular epidermis. The stratum corneum of the scalp represents a physical barrier to the prevention of water loss and the ingress of chemical and/or microbial insults. The quality of the stratum corneum, and hence of the skin itself, is dependent on many factors present within the stratum corneum but the lipidic species which are contained in the water permeability barrier (eg, fatty acids, cholesterol and ceramides) are critical.

The present invention is based on the novel finding that people with dandruff have a weaker scalp barrier function than healthy subjects and that dandruff sufferers are depleted in those lipid species which are comprised in the water permeability barrier in the scalp (eg, ceramides). This observation is true for all people who claim to suffer from dandruff not just those sufferers presenting recognised clinical symptoms of the condition.

Also, it has been found that the scalp of people with dandruff is less able to resist penetration by irritant species. Consequently, dandruff may be treated or prevented from occurring by applying a lipophilic agent to the hair and/or the scalp or by boosting the synthesis of lipids, such as ceramide, within the scalp by applying a lipid precursor and/or promoter to the hair and/or the scalp. The lipophilic agent may specifically ameliorate the quality of the intrinsic scalp barrier or provide a suitable barrier itself, or a lipid precursor and/or promoter may specifically ameliorate the quality of the intrinsic scalp barrier or provide a suitable barrier itself, following its conversion into a lipid. This is in contrast to the teaching in the prior art in which dandruff is said to be treated by suppressing the production of lipid (sebum), hence reducing the level of lipid present on the scalp. The invention is also based on the surprising finding that it is possible to treat dandruff by treating the dandruff affected scalp with a suitable amount of a lipophilic agent, or a lipid precursor and/or promoter and that this precursor and/or promoter, or a suitable derivative, or the lipophilic agent, can be effectively delivered to the scalp from a shampoo.

The present invention involves the recognition that in addition to high levels of Malassezia, scalps affected with dandruff also have a significantly lower level of free intercellular lipids, specifically for example ceramide and cholesterol, than healthy scalps. Treating scalps affected with dandruff, according to a preferred embodiment of the invention, with a combination of an antifungal agent and a lipophilic agent or lipid precursor and/or promoter thus gives superior antidandruff performance.

It has not previously been recognised that total levels of scalp lipids are depleted in scalps affected with dandruff, compared to healthy scalps. Therefore, a solution to the problem of treating dandruff by strengthening the scalp has not been proposed. By treating the scalp with a lipophilic agent or a lipid precursor and/or promoter, initial barrier damage to the scalp is less likely and, therefore, there is less predisposition for damage of the scalp by Malassezia furfur to cause the onset of dandruff.

We have found that dandruff is associated with a dramatic loss of intercellular lipids reflecting underlying problems in epithelial differentiation. Without wishing to be bound by theory, we believe that in the dandruff condition, it is likely that the absence of a fully functional stratum corneum and hence fully functional water permeability barrier will render an individual more prone to further damage from soluble factors derived from surface microbes. Events can therefore be simplified into a cyclical response of decreased barrier performance (either inherent or as a result of Malassezia furfur) leading to increased ingress of microbial derived metabolites, leading to perturbed proliferation/differentiation of keratinocytes in the scalp (causing irritation and inflammation), leading to the production of an inferior stratum corneum, leading to decreased barrier performance, etc. Regular treatment with a lipophilic agent or a lipid precursor and/or promoter, such as by topical application, from a shampoo for example, can prevent this cycle from starting or can help to break it. Also, regular treatment with a lipophilic agent or lipid precursor and/or promoter may make the scalp more resistant to a repeat occurrence of dandruff.

The Lipophilic Agent

The lipophilic agent which is used in the present invention is a substance which is lipid soluble and is capable of being absorbed into the scalp so as to strengthen the scalp.

By the term “strengthen the scalp”, and related terms used herein, we mean that the resistance of the scalp to penetration by moisture is increased ie, the water permeability barrier of the scalp is improved and/or strengthened. The loss of water through the stratum corneum may also be inhibited. Alternative descriptions for the strengthening of the scalp barrier, which are encompassed by the term as used herein, include, but are not limited to, one or more of the following: scalp nourishment, restoring the scalp barrier, strengthening the scalp, helping scalp regenerate, rebuilding the scalp, making smoother scalp skin, soothing the scalp, replenishing the scalp from within, building the scalp, building the scalp from within, building the scalp barrier from within, revitalising the scalp, moisturising the scalp, fully hydrating the scalp. The strengthening of the scalp may be described in certain embodiments of the invention as working with the body.

By strengthening the water permeability barrier of the scalp, the resistance of the scalp to penetration by irritants, such as Malassezia and/or microbial metabolites, is increased. The strengthening of the scalp may include an increase in the physical strength of the body of the scalp (such as increased elasticity) and/or an increase in the ability of the surface of the scalp to act as a barrier in repelling irritants (eg, by becoming more hydrophobic). Thus, the lipophilic agent preferably acts as a so-called moisturising agent for the scalp. The strengthening of the scalp may comprise enhanced epithelial differentiation in the scalp and/or an increased quality of the stratum corneum.

The lipophilic agent preferably has a Log P value of greater than or equal to 1, more preferably greater than 1.5 (such as greater than 1.7), more preferably greater than 2, even more preferably greater than 2.5. Log P values (wherein P is the n-octanol/water partition coefficient and Log P means Log₁₀ P) can be determined as described in J Sangster, Octanol-water partition coefficients of simple organic compounds, J Phys Chem Ref Data, 18, 1111, 1989, the contents of which are incorporated by reference herein, and are typically determined at 25° C. Preferably, Log P values are measured at 25° C.

The lipophilic agent may be a single compound or a mixture of two or more different compounds. It may be advantageous to use a mixture of different compounds as the lipophilic agent, in order to optimise the treatment of the scalp.

The lipophilic agent is preferably used in the invention in the form of a hair treatment composition, such as a shampoo. Typically, the lipophilic agent is present in the shampoo in an amount of from about 0.001% to about 20% by weight, preferably from about 0.01 to about 10% by weight, more preferably from about 1 to about 5% by weight.

A wide variety of lipid type materials and mixtures of materials are suitable for use as the lipophilic agent in the anti-dandruff compositions of the present invention. Preferably, the lipophilic agent is selected from the group consisting of hydrocarbon oils and waxes, fatty acid derivatives, cholesterol, cholesterol derivatives, di- and tri-glycerides, vegetable oils, vegetable oil derivatives, liquid non-digestible oils or blends of liquid digestible or non-digestible oils with solid polyol and acetoglyceride esters, alkyl esters, alkenyl esters, lanolin and its derivatives, milk-tri-glycerides, wax esters, beeswax derivatives, sterols, phospholipids and mixtures thereof.

Suitable hydrocarbon oils and waxes include, for example, petrolatum, mineral oil micro-crystalline waxes, polyalkenes (eg, hydrogenated and nonhydrogenated polybutene and polydecene), paraffins, cerasin, ozokerite, polyethylene and perhydrosqualene. Blends of petrolatum and hydrogenated and non-hydrogenated high molecular weight polybutenes wherein the ratio of petrolatum to polybutene ranges from about 90:10 to about 40:60 are also suitable for use as the lipophilic agent in the compositions described herein.

Preferred di- and tri-glycerides include, for example, castor oil, soy bean oil, derivatized soyabean oils such as maleated soy bean oil, safflower oil, sunflower oil, cotton seed oil, corn oil, walnut oil, peanut oil, coriander seed oil, rapeseed oil, evening primrose oil, passion flower oil, avocado oil, sesame oil, vegetable oils and vegetable oil derivatives, coconut oil and derivatized coconut oil, cottonseed oil, and derivatized cottonseed oil, jojoba oil, cocoa butter, and the like. Sunflower seed oil has been found to be particularly effective.

Suitable acetoglyceride esters are, for example, acetylated monoglycerides.

Lanolin and its derivatives are preferred liphophilic agents for use in the invention. Examples are lanolin, lanolin oil, lanolin wax, lanolin alcohols, lanolin fatty acids, isopropyl lanolate, acetylated lanolin, acetylated lanolin alcohols, lanolin alcohol linoleate and lanolin alcohol riconoleate.

Other suitable lipophilic agents are the ceramides, neoceramides and pseudoceramides.

Suitable ceramides, neoceramides and pseudoceramides for use in the invention are, for example, described in EP-A-0747042, the contents of which are incorporated by reference herein.

Suitable ceramides are, for example, those which are naturally occurring and can be isolated from a suitable plant source or from animal tissue such as pig skin or neural tissue. Ceramides can also be synthesised, as described in EP-A-0747042.

Ceramides are preferably selected from ceramides having structure (I):

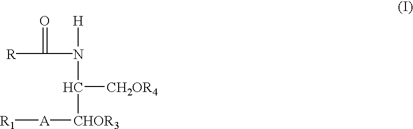

where A represents —CH₂—:—CHOH—; or —CH═CH—

R is a linear or branched, saturated or unsaturated, aliphatic hydrocarbon group having from 1 to 10 carbon atoms which may contain a hydroxyl group;

R₁ represents a linear or branched, saturated or unsaturated, hydroxylated or non-hydroxylated aliphatic hydrocarbon group having from 8 to 28 carbon atoms or a phenyl group;

R₃ represents H, a phosphate group or a sulphate group;

R₄ represents H, a phosphate group, a sulphate group or a sugar group.

Further identification of ceramide structures may be found in U.S. Pat. No. 4,950,688 (Bowser et al) herein incorporated by reference.

Pseudoceramides (i.e. synthetic ceramide-like structures) are preferably selected from pseudoceramides having the general structure (II):

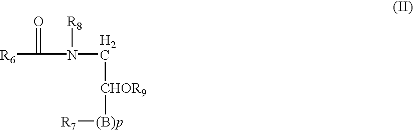

where B represents —OCH₂— or —(CH₂CHOH)— or —CH₂—;

R₆ represents a linear or branched, saturated or unsaturated, or hydroxylated aliphatic hydrocarbon group having from 1 to 10 carbon atoms or a subgroup as described in EP-A-0747042;

R₇ represents a linear or branched, saturated or unsaturated or hydroxylated hydrocarbon group having from 8 to 28 carbon atoms or a phenyl group;

R₈ represents H or a subgroup —(CH₂)_{c}R₁₀, or a subgroup having the structure (A), where c is an integer of from 1 to 6, R₁₀ is —OH or a phosphate group or a sulfate group, or a sugar group;

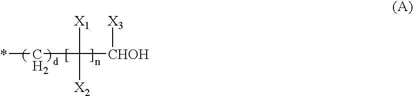

where X₁, X₂ and X₃ each individually represent H, C₁₋₅ alkyl or C₁₋₅ hydroxyalkyl:

n is 1;

d is 0 or an integer from 1 to 4; and

p is 0 or 1;

R₉ represents H, a phosphate group, a sulphate group or a sugar group.

Pseudoceramides may be synthesized according to the procedure described in U.S. Pat. No. 4,778,823 or U.S. Pat. No. 5,198,210 or U.S. Pat. No. 5,206,020, all of which are incorporated by reference herein.

Preferably, in order to attain synergy and minimize cost, pseudoceramides are employed wherein R₈ is CH₂CH₂OH, R₉ is hydrogen, B is —OCH₂— or —CH₂— and R₇ contains from 10 to 22 carbon atoms.

Neoceramides, like pseudoceramides, are synthetic ceramide-like structures. Neoceramides, however, contain more localized polar groups than pseudoceramides. Neoceramides are selected from neoceramides having the general structure (III):

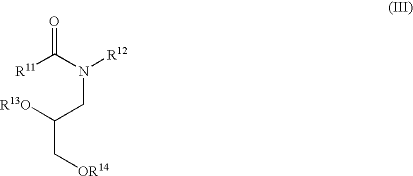

where R¹¹ is a linear or branched, saturated or unsaturated, aliphatic hydrocarbon having from 1 to 10 carbon atoms which may contain a hydroxy group, ester group and/or an ether group; R¹² is a linear branched, saturated or unsaturated aliphatic hydrocarbon group having from 7 to 48 carbon atoms; R¹³ and R¹⁴ are the same or different and each is selected from the group consisting of hydrogen, a sulfate group, a phosphate group, or a sugar group.

Specific preferred examples of ceramides, pseudoceramides, and neoceramides are those described in EP-A-0747072.

Pseudoceramides include, for example, polyol fatty acid polyesters. Polyol fatty acid polyesters are fatty acid polyesters derived from any aliphatic or aromatic polyol which has at least 4 free hydroxyl groups, in which at least 60% of these free hydroxyl groups are esterified with one or more fatty acids having from 8 to 22 carbon atoms. The polyol from which the polyol fatty acid polyesters are derived are preferably chosen from sugar polyols, which include mono-, di- and polysaccharides. Preferred examples of monosaccharide sugar polyols include pentose sugar polyols, hexose sugar polyols and heptose sugar polyols. Pentose sugar polyols include, for example, D-ribose, D-arabinose, D-xylose, D-lyxose, D-ribulose and D-xylulose. Hexose sugar polyols include, for example, D-allose, D-altrose, D-glucose, D-mannose, D-gulose, D-idose, D-galactose, D-talose, D-fructose, D-sorbose and D-tagatose. Heptose sugar polyols include, for example, D-mannoheptulose and D-sedoheptulose.

The polyol from which the polyol fatty acid polyester is derived can also be chosen from: disaccharides such as maltose, lactose, cellobiose, sucrose, trehalose, gentiobiose, melibiose and primeverose; tri-saccharides, such as gentianose and raffinose; sugar alcohols such as D-mannitol, D-sorbitol, D-ribitol, D-erithritol, D-lactitol and D-xylitol; and derivatives of sugars such as methyl glucoside and inositol.

The preferred sugar polyol is sucrose.

The fatty acids which are employed to form the polyol fatty acid polyesters of the invention can be individual free fatty acids having from 8 to 22 carbon atoms in the fatty acid molecule. These fatty acids can be saturated or unsaturated, linear or branched chain fatty acids.

Preferred sources of fatty acids for forming the polyol fatty acid polyesters are naturally occurring fats and oils which provide a source of a blend of fatty acid residues. Selection of the particular source of fatty acids can vary widely the physical and chemical properties of the polyol fatty acid polyesters obtained therefrom.

The naturally occurring fats and oils can be directly obtained from nature or can be derivatives thereof, for example following full or partial hydrogenation, interesterification, transfection or fractionation.

Suitable natural sources of fatty acid residues include those of animal, marine or vegetable origin, such as tallow, lanolin oil, cod liver oil, halibut liver oil, other fish oils, coconut oil, palmkernel oil, palm oil, butter fat, soyabean oil, safflower oil, cotton seed oil, rapeseed oil, poppy seed oil, corn oil, sunflower oil, groundnut oil and mixtures thereof. Preferred fatty acid sources are palm oils, partially hydrogenated palm oils, palmkernel oils, optionally partially hydrogenated soya bean oils and partially hydrogenated fish oils.

By employing a mixture of fatty acids, or one or more naturally occurring oils such as those exemplified above, when synthesising the polyol fatty acid polyester, it is possible to provide polyol fatty acid polyesters in which a mixture of ester groups is present on a single polyol molecule. In this way it is possible to vary the melting characteristics of the polyol fatty acid polyester so formed, as desired.

Particularly preferred polyol fatty acid polyesters are sucrose fatty acid polyesters where the ester is derived from lauric acid or natural oils, such as palm oil, palm kernel oil, soya bean oil, coconut oil, fish oil and mixtures thereof.

The Lipid Precursor and/or Promoter

The lipid precursor and/or promoter which is used in the present invention may be a precursor of a lipid or may promote the formation of a lipid or may act both as a precursor of a lipid and to promote the formation of the same or another lipid.

The lipid precursors which may used in the present invention are substances which are capable of being absorbed into the scalp so as to strengthen the scalp, by being converted to a lipid under the action of one or more natural biochemical processes. The lipid formed from the precursor is a compound which may typically be naturally present in the stratum corneum, for example a ceramide.

The lipid promoters which may used in the present invention are substances which are capable of being absorbed into the scalp so as to strengthen the scalp, by promoting the conversion of lipid precursors to lipid under the action of one or more natural biochemical processes. The promoters may promote or enhance the activity of an enzyme which is involved in the synthesis of lipids, or may promote the formation of lipids by another mechanism.

The lipid precursor and/or promoter is preferably used in the invention in the form of a hair treatment composition, such as a shampoo. Typically, the lipid precursor and/or promoter is present in the shampoo in an amount of from about 0.01% to about 20% by weight, preferably from about 0.1% to about 10% by weight, more preferably from about 0.1% to about 5% by weight, most preferably from about 0.1% to about 1% by weight.

The lipid precursor and/or promoter is preferably selected from alpha hydroxy acids (AHAs), beta-hydroxy acids (BHAs), omega-hydroxy acids, serine palmitoyl CoA transferase activators, ceramide precursors and mixtures thereof.

The lipid precursor and/or promoter may be a single compound or a mixture of two or more different compounds. It may be advantageous to use a mixture of different compounds as the lipid precursor and/or promoter, in order to optimise the treatment of the scalp. Thus, the compositions of the invention may comprise two or more lipid precursor and/or promoters. Preferably, at least one of the lipid precursor and/or promoters is selected from one of the following groups:

(i) alpha hydroxy acids (AHAs);

(ii) beta-hydroxy acids (BHAs);

(iii) omega-hydroxy acids;

(iv) serine palmitoyl CoA transferase activators; and

(v) ceramide precursors

and at least one other lipid precursor and/or promoter is selected from a different one of said groups (i) to (v). More preferably, the composition of the invention comprises at least one compound from group (v) and at least one compound from groups (i) to (iii).

Alpha Hydroxy Acids (AHAs)

AHAs are well known in the literature for use in the treatment of dry/flaky skin, fine lines and wrinkles. Studies have shown that AHAs operate via five different mechanisms:

1. Water binding—AHAs bind water due to the presence of the hydrophilic hydroxyl groups which enhances the plasticization of the skin.

2. Desquamation—AHAs are very effective at treating very dry skin conditions; the precise mechanism is not known although it has been proposed that this is due to reduced corneocyte cohesion as a result of interference in ionic bonding by the AHA.

3. Keratinocyte activity—AHAs impact on both proliferation and differentiation.

4. Dermal protein synthesis.

5. Stratum corneum ceramide biosynthesis.

The alpha-hydroxy carboxylic acids are represented by formula I having the structure:

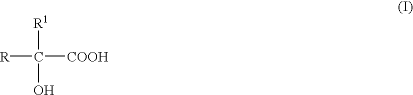

wherein R and R¹ may be the same or different and are selected from H, F, Cl, Br, alkyl, aralkyl or aryl groups which may be saturated or unsaturated, isomeric or nonisomeric, straight or branched chain, having 1 to 25 carbon atoms, or in a cyclic form having 5 or 6 ring members. In addition, R and R¹ may be substituted with an OH, CHO, COOH or alkoxy group having 1 to 9 carbon atoms. The alpha-hydroxy acid exists as a free acid, and includes stereoisomers, and D, L, and DL forms thereof when R and R¹ are not identical.

Illustrative of this group of materials are: 2-hydroxyethanoic acid (glycolic acid); 2-hydroxypropanoic acid (lactic acid); 2-methyl 2-hydroxypropanoic acid (methyllactic acid); 2-hydroxybutanoic acid; 2-hydroxypentanoic acid; 2-hydroxyhexanoic acid; 2-hydroxyheptanoic acid; 2-hydroxyoctanoic acid; 2-hydroxynonanoic acid; 2-hydroxydecanoic acid; 2-hydroxyundecanoic acid; 2-hydroxydodecanoic acid (alpha-hydroxylauric acid); 2-hydroxytetradecanoic acid (alpha-hydroxymyristic acid); 2-hydroxyhexadecanoic acid (alpha-hydroxypalmitic acid); 2-hydroxyoctadecanoic acid(alpha-hydroxystearic acid); 2-hydroxyeicosanoic acid (alpha-hydroxyarachidonic acid); 2-phenyl 2-hydroxyethanoic acid (mandelic acid); 2,2-diphenyl 2-hydroxyethanoic acid (benzilic acid); 3-phenyl 2-hydroxypropanoic acid (phenyl lactic acid); 2-phenyl, 2-methyl, 2-hydroxyethanoic acid (atrolactic acid); 2-(4′-hydroxyphenyl) 2-hydroxyethanoic acid; 2-(4′-chlorophenyl), 2-hydroxyethanoic acid; 2-(3′-hydroxy-4′-methoxyphenyl) 2-hydroxyethanoic acid; 2-(4′-hydroxy-3′-methoxyphenyl)2-hydroxyethanoic acid; 3′-(2-hydroxyphenyl) 2-hydroxypropanoic acid; 3-(4′-hydroxyphenyl) 2-hydroxypropanoic acid; and 2-(3′,4′-dihydroxyphenyl) 2-hydroxyethanoic acid.

The most preferred alpha-hydroxy carboxylic acids are glycolic acid, lactic acid or 2-hydroxyoctanoic acid, and mixtures thereof.

Beta Hydroxy Acids

Beta-hydroxy acids are compounds containing a hydroxy group bonded to the carbon atom which is adjacent to the carbon atom bonded to the carboxylic acid (COOH) group. Preferably, the two aforesaid carbon atoms may form part of an aromatic (eg, optionally substituted phenyl) ring or may be part of a straight or branched chain alkyl or alkylene group having from 2 to 24 carbon atoms.

Suitable beta-hydroxy acids for use in the present invention include, for example, salicylic acid and its derivatives (eg, esters and cosmetically acceptable salts therof, such as alkali metal and ammonium salts).

Omega Hydroxy Acids

The omega-hydroxy carboxylic acids are represented by compounds having the formula HO—R′—COOH, wherein R′ is selected from alkylene, alkenylene, aralkylene or arylene groups which may be saturated or unsaturated, isomeric or nonisomeric, straight or branched chain, having 1 to 25 carbon atoms, or in a cyclic form having 5 or 6 ring members. In addition, R′ may be substituted with an OH, CHO, COOH or alkoxy group having 1 to 9 carbon atoms. Preferably, R′ is straight chain alkylene having from 3 to 24 carbon atoms, more preferably from 12 to 24 carbon atoms; such acids may be termed omega fatty acids. The omega-hydroxy acid may exist as a free acid, a cosmetically acceptable salt, such as an alkali metal or ammonium salt, or a lactone.

Preferred omega-hydroxy acids include juniperic acid and its lactone.

Serine Palmitoyl CoA Transferase Activators

Serine palmitoyl CoA transferase activators include, for example, thiol or S-ester plus L-serine or N-acetyl cysteine, alpha-lipoic acid, S-lactoyl glutathione, mercaptosuccinic acid, thiosalicylic acid, thioglycerin and cosmetically acceptable salts thereof.

Suitable serine palmitoyl CoA transferase activators are described in U.S. Pat. No. 5,472,698 which teaches that a combination of a thiol, disulphide or S-ester plus L-serine or N-acetyl serine can be used to increase sphingolipid production in the skin and improving water barrier performance. The contents of this document are incorporated by reference herein.

Suitable thiol compounds include cystamine, cysteamine, N-acetylcysteamine, N-acetyl-L-cysteine, 6,8-thioctic acid, thioacetamide, thioacetanilide, o-thiocresol, m-thiocresol, p-thiocresol, 6-thioctic acid, thioctic amide, thiodiacetic acid, thiodiglycolic acid, thiosalicylic acid, thiogalactoside, thiodiglucoside, 3,3′-thiodipropionic acid, thioglycolic acid, 1-thio-beta-D-glucose, thiourea, pantetheine, dithioerythritol, 1,4-dithio-L-threitol, mercaptoacetic acid, 2-mercaptobenzimidazole, 2-mercaptobenzothiazole, 2-mercaptobenzoxazole, 2-mercapto-2-butanol, 2-mercaptoethanol, 2-mercaptoethyl sulfide, 2-mercapto-imidazole, 2-mercaptomenthone, 2-mercaptonicotinic acid, 2-mercapto-5-nitrobenzimidazole, 3-mercapto-1-propanediol, 2-mercaptopropionic acid, 3-mercaptopropionic acid, N-(2-mercaptopropionyl)glycine, 2-mercaptopyrimidine, mercaptosuccinic acid, 2,3-dimercapto-1-propanesulfonic acid, 2,3-dimercapto-1-propanol, 2,3-dimercapto-1-propanol tributyrate, meso-2,3-dimercaptosuccinic acid, 3,4-dimercaptotoluene. Preferred are N-acetyl-L-cysteine and thioctic acid.

Suitable compounds containing an S-ester group include, for example, cysteine S-esters (eg, S-acetamidomethyl-L-cysteine, (S)-2-aminoethyl-L-cysteine, S-benzyl-L-cysteine and its methyl or ethyl esters, S-t-butylmercapto-L-cysteine, S-carbamyl-L-cysteine, S-ethyl-L-cysteine, S-methyl-L-cysteine, S-lactoyl-cysteine, S-hydroxycaproyl-cysteine), S-adenosylmethionine, coenzyme A derivatives (eg, N,S-diacetyl-beta-mercaptoethylamine), S-esters of glutathione (eg, S-lactoyl glutathione), S-butylglutathione, S-methylglutathione, S-decylglutathione, S-ethylglutathione, S-heptylglutathione, S-hexylglutathione, S-nonylglutathione, S-octylglutathione, S-pentylglutathione, S-propylglutathione.

Examples of suitable disulphides include N-acyl or hydroxyacyl derivatives of cystine and 2,2′-dithiosalicylic acid.

Ceramide Precursors

The basic ceramide structure consists of a fatty acid covalently bound to a long chain sphingoid base. In human stratum corneum ceramides, the sphingoid bases present are sphingosine and phytosphingosine. A derivative of phytosphingosine is tetraacetyl phytosphingosine (TAPS).

Ceramide precursors therefore include, for example, tetraacetyl phytosphingosine (TAPS), phytosphingosine, phycosine and sphingomyelin. Other ceramide precursors include alkyl and alkenyl carboxylic acids containing from 12 to 24 carbom atoms, for example palmitic acid and linoleic acid.

Antifungal Agents

The lipophilic agents or lipid precursor and/or promoters are preferably used in the invention in conjunction with an antifungal agent. When used in conjunction with an antifungal agent, the lipophilic agent or lipid precursor and/or promoters may be applied to the hair and/or the scalp at the same time as, before or after, treatment with the antifungal agent. Preferably, the lipophilic agent or lipid precursor and/or promoter agent and the antifungal agent are applied to the hair and/or the scalp at the same time from a single composition.

The preferred antifungal agent is zinc pyrithione (ZnPTO) which, on account of its relative insolubility in aqueous systems, is generally used in hair treatment compositions as a particulate dispersion. The zinc pyrithione may be used in any particle form including, for example, crystalline forms such as platelets and needles and amorphous, regularly or irregularly shaped particles. If zinc pyrithione is present in the composition, a suspending agent is preferably used to prevent or inhibit the settling of the particles out of the composition. The average particle diameter of the zinc pyrithione particles (ie, their maximum dimension) is typically from about 0.2 to about 50 μm, preferably from about 0.4 to about 10 μm, such as about 0.1 to about 5 μm, more preferably from 0.1 μm to 1 μm as determined, for example, using a Malvern Mastersizer (Malvern Instruments, UK).

Other preferred anti-dandruff agents include climbazole, ketoconazole and octopirox. Anti-dandruff agents may be used alone or as mixtures of one or more such agents.

Antifungal agents typically display a minimum inhibitory concentration of about 50 mg/ml or less against Malassezia.

If the antifungal agent is soluble in aqueous systems, it may be present in solution in a composition used in the invention.

The antifungal agent may be present in the composition in an amount of from 0.001% to 10% by weight, preferably from 0.1% to 5% by weight, more preferably from 0.5% to 3% by weight.

Method of the Invention

The method of the invention preferably comprises the steps of:

(a) contacting the scalp with water;

(b) applying to the scalp a lipophilic agent or lipid precursor and/or promoter in an amount effective to penetrate the stratum corneum;

(c) rinsing said lipophilic agent or lipid precursor and/or promoter from the scalp; and

(d) determining the extent to which the dandruff has been treated.

Steps (a), (b) (c) are typically carried out in an analogous manner to the conventional treatment of hair by shampooing and/or conditioning. Step (d) may involve a determination by the user of the product by visual observation and/or by sensing the degree to which the symptoms of the dandruff (such as scalp itch) have been alleviated or eliminated. Alternatively, step (d) may involve a physical measurement of the extent of flaking skin on the scalp (eg, by removing flakes of skin onto an adhesive substrate such as a strip of adhesive tape) and/or of the extent to which the scalp has been strengthened, for example as measured either by corneosurfametry (CSM), transepidermal water loss (TEWL), corneometry or by measuring the amount of stratum corneum lipid (e.g. ceramide or cholesterol) per weight of protein present in the stratum corneum.

Product Forms

Compositions useful in the present invention may be formulated as transparent or opaque emulsions, lotions, creams, pastes or gels. Particularly preferred product forms are shampoos and conditioners, especially shampoos. Compositions according to the invention, such as shampoo and conditioner compositions, will typically contain water in an amount of from about 50% to about 98% by weight, preferably from about 60% to about 90% by weight, most preferably at least 70% by weight.

Shampoo Compositions

A particularly preferred hair treatment composition in accordance with the invention is a shampoo composition.

Such a shampoo composition will comprise one or more cleansing surfactants which are cosmetically acceptable and suitable for topical application to the hair. Further surfactants may be present as an additional ingredient if sufficient for cleansing purposes is not provided as emulsifier for any emulsified components in the composition, e.g. emulsified silicones. It is preferred that shampoo compositions of the invention comprise at least one further surfactant (in addition to that used as emulsifying agent) to provide a cleansing benefit.

Suitable cleansing surfactants, which may be used singularly or in combination, are selected from anionic, amphoteric and zwitterionic surfactants, and mixtures thereof. The cleansing surfactant may be the same surfactant as the emulsifier, or may be different.

Examples of anionic surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule.

Typical anionic surfactants for use in shampoos of the invention include sodium oleyl succinate, ammonium lauryl sulphosuccinate, ammonium lauryl sulphate, sodium dodecylbenzene sulphonate, triethanolamine dodecylbenzene sulphonate, sodium cocoyl isethionate, sodium lauryl isethionate and sodium N-lauryl sarcosinate. The most preferred anionic surfactants are sodium lauryl sulphate, triethanolamine monolauryl phosphate, sodium lauryl ether sulphate 1 EO, 2EO and 3EO, ammonium lauryl sulphate and ammonium lauryl ether sulphate 1EO, 2EO and 3EO.

Examples of amphoteric and zwitterionic surfactants include alkyl amine oxides, alkyl betaines, alkyl amidopropyl betaines, alkyl sulphobetaines (sultaines), alkyl glycinates, alkyl carboxyglycinates,alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine and sodium cocamphopropionate.

The shampoo composition can also include co-surfactants, to help impart aesthetic, physical or cleansing properties to the composition. A preferred example is a nonionic surfactant, which can be included in an amount ranging from 0% to about 5% by weight of the total composition.

For example, representative nonionic surfactants that can be included in shampoo compositions of the invention include condensation products of aliphatic (C₈-C₁₈) primary or secondary linear or branched chain alcohols or phenols with alkylene oxides, usually ethylene oxide and generally having from 6 to 30 ethylene oxide groups.

Other representative nonionics include mono- or di-alkyl alkanolamides. Examples include coco mono- or diethanolamide and coco mono-isopropanolamide.

Further nonionic surfactants which can be included in shampoo compositions of the invention are the alkyl polyglycosides (APGs). Typically, the APG is one which comprises an alkyl group connected (optionally via a bridging group) to a block of one or more glycosyl groups. Preferred APGs are defined by the following formula:

RO−(G)ₙ

wherein R is a branched or straight chain alkyl group which may be saturated or unsaturated and G is a saccharide group.

R may represent a mean alkyl chain length of from about C₅ to about C₂₀. Preferably R represents a mean alkyl chain length of from about C₈ to about C₁₂. Most preferably the value of R lies between about 9.5 and about 10.5. G may be selected from C₅ or C₆ monosaccharide residues, and is preferably a glucoside. G may be selected from the group comprising glucose, xylose, lactose, fructose, mannose and derivatives thereof. Preferably G is glucose.

The degree of polymerisation, n, may have a value of from about 1 to about 10 or more. Preferably, the value of n lies in the range of from about 1.1 to about 2. Most preferably the value of n lies in the range of from about 1.3 to about 1.5.

Suitable alkyl polyglycosides for use in the invention are commercially available and include for example those materials identified as: Oramix NS10 ex Seppic; Plantaren 1200 and Plantaren 2000 ex Henkel.

The total amount of surfactant (including any co-surfactant, and/or any emulsifier) in shampoo compositions of the invention is generally from 0.1 to 50% by weight, preferably from 5 to 30%, more preferably from 10% to 25% by weight of the total shampoo composition.

A cationic deposition polymer is a preferred ingredient in shampoo compositions of the invention, for enhancing conditioning performance of the shampoo. By “deposition polymer” is meant an agent which enhances deposition of the silicone component from the shampoo composition onto the intended site during use, i.e. the hair and/or the scalp.

The deposition polymer may be a homopolymer or be formed from two or more types of monomers. The molecular weight of the polymer (in g/mol) will generally be between 5 000 and 10 000 000, typically at least 10 000 and preferably in the range 100 000 to about 2 000 000. The polymers will have cationic nitrogen containing groups such as quaternary ammonium or protonated amino groups, or a mixture thereof.

The cationic nitrogen-containing group will generally be present as a substituent on a fraction of the total monomer units of the deposition polymer. Thus when the polymer is not a homopolymer it can contain spacer non-cationic monomer units. Such polymers are described in the CTFA Cosmetic Ingredient Directory, 3rd edition. The ratio of the cationic to non-cationic monomer units is selected to give a polymer having a cationic charge density in the required range.

Suitable cationic deposition polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are preferred.

Amine substituted vinyl monomers and amines can be polymerized in the amine form and then converted to ammonium by quaternization.

The cationic deposition polymers can comprise mixtures of monomer units derived from amine- and/or quaternary ammonium-substituted monomer and/or compatible spacer monomers.

Suitable cationic deposition polymers include, for example:

copolymers of 1-vinyl-2-pyrrolidine and 1-vinyl-3-methyl-imidazolium salt (e.g. chloride salt), referred to in the industry by the Cosmetic, Toiletry, and Fragrance Association, (CTFA) as Polyquaternium-16. This material is commercially available from BASF Wyandotte Corp. (Parsippany, N.J., USA) under the LUVIQUAT tradename (e.g. LUVIQUAT FC 370);

copolymers of 1-vinyl-2-pyrrolidine and dimethylaminoethyl methacrylate, referred to in the industry (CTFA) as Polyquaternium-11. This material is available commercially from Gaf Corporation (Wayne, N.J., USA) under the GAFQUAT tradename (e.g., GAFQUAT 755N);

cationic diallyl quaternary ammonium-containing polymers including, for example, dimethyldiallyammonium chloride homopolymer and copolymers of acrylamide and dimethyldiallylammonium chloride, referred to in the industry (CTFA) as Polyquaternium 6 and Polyquaternium 7, respectively;

mineral acid salts of amino-alkyl esters of homo-and co-polymers of unsaturated carboxylic acids having from 3 to 5 carbon atoms, (as described in U.S. Pat. No. 4,009,256);

cationic polyacrylamides(as described in WO95/22311).

Other cationic deposition polymers that can be used include cationic polysaccharide polymers, such as cationic cellulose derivatives, cationic starch derivatives, and cationic guar gum derivatives.

Cationic polysaccharide polymers suitable for use in compositions of the invention include those of the formula:

A—O—[R—N⁺(R¹)(R²)(R³)X^{-],}

wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Other suitable cationic polysaccharide polymers include quaternary nitrogen-containing cellulose ethers (e.g. as described in U.S. Pat. No. 3,962,418), and copolymers of etherified cellulose and starch (e.g. as described in U.S. Pat. No. 3,958,581).

A particularly suitable type of cationic polysaccharide polymer that can be used is a cationic guar gum derivative, such as guar hydroxypropyltrimonium chloride (Commercially available from Rhodia (formerly Rhone-Poulenc) in their JAGUAR trademark series).

Examples are JAGUAR C13S, which has a low degree of substitution of the cationic groups and high viscosity. JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution. Preferably the cationic deposition polymer is selected from cationic cellulose and cationic guar derivatives.

Particularly preferred deposition polymers are JAGUAR C13S, JAGUAR C15, JAGUAR C17 and JAGUAR C16 and JAGUAR C162.

The cationic deposition polymer will generally be present at levels of from 0.001 to 5%, preferably from about 0.01 to 1%, more preferably from about 0.02% to about 0.5% by weight of the total composition.

Solid Active Agents

Solid active agents include the antifungal agents mentioned hereinbefore such as the heavy metal salts of pyridinethione, especially zinc pyridinethione and other antimicrobials such as selenium sulphide.

Other suitable solid active agents include pigment particles, such as solid dyes or colorants suitable for application to hair, and metal colloids.

Aesthetic Agents

Hair treatment compositions such as shampoos and conditioners are frequently opacified or pearlised to enhance consumer appeal.

Examples of opacifying agents include higher fatty alcohols (e.g. cetyl, stearyl, arachidyl and behenyl), solid esters (e.g. cetyl palmitate, glyceryl laurate, stearamide MEA-stearate), high molecular weight fatty amides and alkanolamides and various fatty acid derivatives such as propylene glycol and polyethylene glycol esters. Inorganic materials used to opacify hair treatment compositions include magnesium aluminium silicate, zinc oxide, and titanium dioxide.

Pearlescing agents typically form thin, platelet-type crystals in the composition, which act like tiny mirrors. This gives the pearl lustre effect. Some of the opacifying agents listed above may also crystallise as pearlescing agents, depending on the media in which they are used and the conditions employed.

Typical pearlescing agents may be selected from C16-C22 fatty acids (e.g. stearic acid, myristic acid, oleic acid and behenic acid), esters of C16-C22 fatty acid with alcohols and esters of C16-C22 fatty acid incorporating such elements as alkylene glycol units. Suitable alkylene glycol units may include ethylene glycol and propylene glycol. However, higher alkylene chain length glycols may be employed. Suitable higher alkylene chain length glycols include polyethylene glycol and polypropylene glycol.

Examples are polyethylene glycol mono or diesters of C16-C22 fatty acids having from 1 to 7 ethylene oxide units, and ethylene glycol esters of C16-C22 fatty acids. Preferred esters include polyethylene glycol distearates and ethylene glycol distearates. Examples of a polyethylene glycol distearate available commercially are EUPERLAN PK900 (ex Henkel) or GENAPOL TS (ex Hoechst). An example of an ethylene glycol distearate is EUPERLAN PK3000 (ex Henkel).

Other pearlescing agents include alkanolamides of fatty acids having from 16 to 22 carbon atoms, (e.g. stearic monoethanolamide, stearic diethanolamide, stearic monoisopropanolamide and stearic monoethanolamide stearate); long chain esters of long chain fatty acids (e.g. stearyl stearate, cetyl palmitate); glyceryl esters (e.g. glyceryl distearate),long chain esters of long chain alkanolamides (e.g. stearamide DEA distearate, stearamide MEA stearate), and alkyl (C18-C22) dimethyl amine oxides (e.g. stearyl dimethyl amine oxide).

Further suitable pearlescing agents include inorganic materials such as nacreous pigments based on the natural mineral mica. An example is titanium dioxide coated mica. Particles of this material may vary in size from 2 to 150 microns in diameter. In general, smaller particles give rise to a pearly appearance, whereas particles having a larger average diameter will result in a glittery composition.

Suitable titanium dioxide coated mica particles are those sold under the trade names TIMIRON (merck) or FLAMENCO (Mearl).

The level of opacifying or pearlescing agent employed in compositions of the invention is generally from 0.01 to 20%, preferably 0.01 to 5%, more preferably from 0.02 to 2% by weight of the total composition.

Gas (e.g. air) bubbles represent another type of suspended phase that may be introduced into a hair treatment composition for aesthetic purposes. When evenly sized and homogeneously dispersed in the composition, these can enhance consumer appeal—a typical application is in a transparent or translucent composition such as a hair styling gel.

Conditioners

Compositions in accordance with the invention may also be formulated as conditioners for the treatment of hair (typically after shampooing) and subsequent rinsing.

Such a conditioner will comprise one or more conditioning surfactants which are cosmetically acceptable and suitable for topical application to the hair.

Suitable conditioning surfactants are selected from cationic surfactants, used singly or in admixture. Examples include quaternary ammonium hydroxides or salts thereof, e.g chlorides.

Suitable cationic surfactants for use in hair conditioners of the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in hair conditioners of the invention is cetyltrimethylammonium chloride, available commercially, for example as GENAMIN CTAC, ex Hoechst Celanese.

In conditioners of the invention, the level of cationic surfactant is preferably from 0.01 to 10%, more preferably 0.05 to 5%, most preferably 0.1 to 2% by weight of the composition.

Conditioners of the invention advantageously incorporate a fatty alcohol. The combined use of fatty alcohols and cationic surfactants in conditioning compositions is believed to be especially advantageous, because this leads to the formation of a lamellar phase, in which the cationic surfactant is dispersed.

Representative fatty alcohols comprise from 8 to 22 carbon atoms, more preferably 16 to 20. Examples of suitable fatty alcohols include cetyl alcohol, stearyl alcohol and mixtures thereof. The use of these materials is also advantageous in that they contribute to the overall conditioning properties of compositions of the invention.

The level of fatty alcohol in conditioners of the invention is conveniently from 0.01 to 10%, preferably from 0.1 to 5% by weight of the composition. The weight ratio of cationic surfactant to fatty alcohol is suitably from 10:1 to 1:10, preferably from 4:1 to 1:8, optimally from 1:1 to 1:4.

Conditioning Agents

The compositions which may be used in the invention may contain a conditioning agent. As used herein, the term “conditioning agent” includes any material which is used to give a particular conditioning benefit to hair and/or skin. For example, in compositions for use in washing hair, such as shampoos and conditioners, suitable materials are those which deliver one or more benefits relating to shine, softness, combability, wet-handling, anti-static properties, protection against damage, body, volume, stylability and manageability.

Preferred conditioning agents for use in the present invention include emulsified silicones, used to impart for example wet and dry conditioning benefits to hair such as softness, smooth feel and ease of combability.

Various methods of making emulsions of particles of silicones for use in the invention are available and are well known and documented in the art.

The viscosity of the silicone itself (not the emulsion or the final washing composition) preferably ranges from 10,000 cps to 5 million cps. The viscosity can be measured by means of a glass capillary viscometer as set out further in Dow Corning Corporate Test Method CTM004 Jul. 20 1970.

Suitable silicones include polydiorganosiloxanes, in particular polydimethylsiloxanes which have the CTFA designation dimethicone. An example is dimethicone fluid having a viscosity of up to 100,000 centistokes at 25° C., which is available commercially from the General Electric Company as the Viscasil series and from Dow Corning as the DC₂₀₀ series.

Aminofunctional silicones which have the CTFA designation amodimethicone, are also suitable for use in the compositions of the invention, as are polydimethyl siloxanes having hydroxyl end groups, which have the CTFA designation dimethiconol.

Also suitable are silicone gums. “Silicone gum” denotes polydiorganosiloxanes having a molecular weight of from 200,000 to 1,000,000 g/mol and specific examples include dimethicone gums, dimethiconol gums, polydimethyl siloxane/diphenyl/methylvinylsiloxane copolymers, polydimethylsiloxane/methylvinylsiloxane copolymers and mixtures thereof. Examples include those materials described in U.S. Pat. No. 4,152,416 (Spitzer), and on General Electric Silicone Rubber product Data Sheet SE 30, SE 33, SE 54 and SE 76.

Also suitable for use in the present invention are silicone gums having a slight degree of cross-linking, as are described for example in WO 96/31188. These materials can impart body, volume and stylability to hair, as well as good wet and dry conditioning.

Preferred emulsified silicones for use in compositions of the invention have an average silicone particle size in the composition of less than 100, preferably less than 30, more preferably less than 20 microns, most preferably less than 10 microns.

Particle size may be measured by means of a laser light scattering technique, using a 2600D Particle Sizer from Malvern Instruments.

Suitable silicone emulsions for use in the invention are commercially available in a pre-emulsified form. This is particularly preferred since the pre-formed emulsion can be incorporated into the washing composition by simple mixing.

Examples of suitable pre-formed emulsions include emulsions DC2-1766 and DC2-1784, available from Dow Corning. These are emulsions of dimethiconol. Cross-linked silicone gums are also available in a pre-emulsified form, which is advantageous for ease of formulation. A preferred example is the material available from Dow Corning as DC X2-1787, which is an emulsion of cross-linked dimethiconol gum.

The amount of silicone incorporated into the compositions of the invention depends on the level of conditioning desired and the material used. A preferred amount is from 0.01 to about 10% by weight of the total composition although these limits are not absolute. The lower limit is determined by the minimum level to achieve conditioning and the upper limit by the maximum level to avoid making the hair and/or skin unacceptably greasy. We have found that an amount of silicone of from 0.5 to 1.5% by weight of the total composition, is a particularly suitable level.

A further preferred class of conditioning agents are per-alk(en)yl hydrocarbon materials, used to enhance the body, volume and stylability of hair.

EP 567 326 and EP 498 119 describe suitable peralk(en)yl hydrocarbon materials for imparting stylability and enhanced body to hair. Preferred materials are polyisobutylene materials available from Presperse, Inc. under the PERMETHYL trade name.

The amount of per-alk(en)yl hydrocarbon material incorporated into the compositions of the invention depends on the level of body and volume enhancement desired and the specific material used. A preferred amount is from 0.01 to about 10% by weight of the total composition although these limits are not absolute. The lower limit is determined by the minimum level to achieve the body and volume enhancing effect and the upper limit by the maximum level to avoid making the hair unacceptably stiff. We have found that an amount of per-alk(en)yl hydrocarbon material of from 0.5 to 2% by weight of the total composition is a particularly suitable level.

When the hydrocarbon material is lipophilic, it may constitute all or part of the lipophilic agent of the invention.

Optional Ingredients

Compositions of this invention may contain any other ingredient normally used in hair treatment formulations. These other ingredients may include viscosity modifiers, preservatives, colouring agents, polyols such as glycerine and polypropylene glycol, chelating agents such as EDTA, antioxidants, fragrances, and sunscreens. Each of these ingredients will be present in an amount effective to accomplish its purpose. Generally these optional ingredients are included individually at a level of up to about 5% by weight of the total composition.

Preferably, compositions of this invention also contain adjuvants suitable for hair care. Generally such ingredients are included individually at a level of up to 2%, preferably up to 1%, by weight of the total composition.

Among suitable hair care adjuvants, are:

(i) natural hair root nutrients, such as amino acids and sugars. Examples of suitable amino acids include arginine, cysteine, glutamine, glutamic acid, isoleucine, leucine, methionine, serine and valine, and/or precursors and derivatives thereof. The amino acids may be added singly, in mixtures, or in the form of peptides, e.g. di- and tripeptides. The amino acids may also be added in the form of a protein hydrolysate, such as a keratin or collagen hydrolysate. Suitable sugars are glucose, dextrose and fructose. These may be added singly or in the form of, e.g. fruit extracts. A particularly preferred combination of natural hair root nutrients for inclusion in compositions of the invention is isoleucine and glucose. A particularly preferred amino acid nutrient is arginine.

(ii) hair fibre benefit agents. Examples are:

ceramides, for moisturising the fibre and maintaining cuticle integrity. Ceramides are available by extraction from natural sources, or as synthetic ceramides and pseudoceramides. A preferred ceramide is Ceramide II, ex Quest. Mixtures of ceramides may also be suitable, such as Ceramides LS, ex Laboratoires Serobiologiques. The ceramides may constitute all or part of the lipophilic agent of the invention.

The invention will now be further illustrated by the following, non-limiting examples. In the examples and throughout the specification all percentages are by weight based on the total weight of the composition, unless indicated otherwise.

### EXAMPLES

The examples include reference to the figures which illustrate the invention.

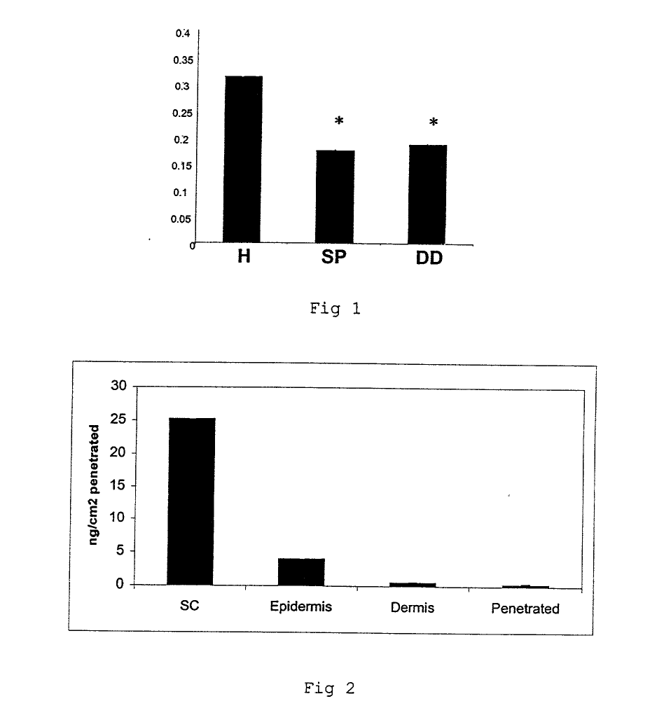

FIG. 1 is a plot showing the relative amount of ceramide in the scalp of a number of subjects.

FIG. 2 shows the penetration of triglyceride from shampoo containing sunflower seed oil and zinc pyrithione (ZnPTO).

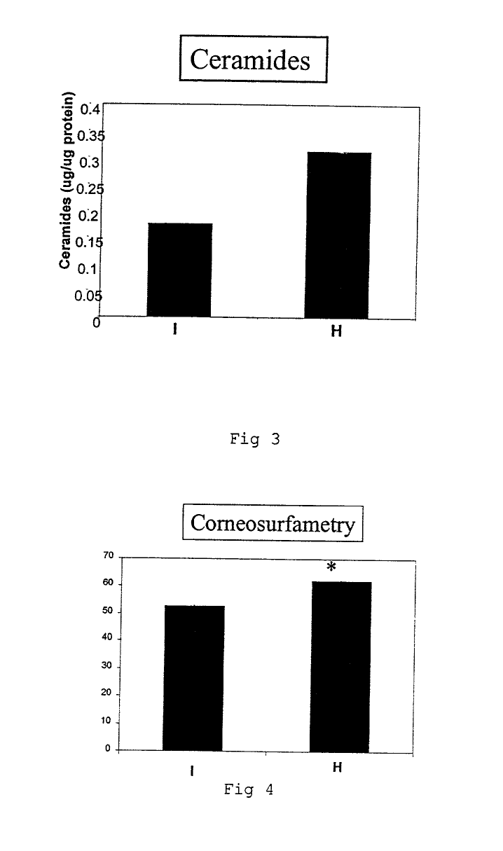

FIG. 3 is a plot showing the relative amount of ceramide in the scalp of a number of subjects, H denotes control subjects having a healthy scalp and I denotes subjects complaining of an itchy scalp.

FIG. 4 is a plot which shows that scalp itch can be accompanied by a reduction in scalp barrier function as measured by corneosurfametry (CSM).

Shampoo and conditioner formulations referred to in the examples may be prepared by methods well-known in the art.

### Example 1

Tests on the ability of compositions to increase ceramide production in skin were carried out. The formulation pairs tested were:

1.0% tetraacetyl phytosphingosine (TAPS) vs Vehicle

1.0% TAPS+1.0% juniperic acid+1.0% linoleic acid vs Vehicle.

Vehicle=ethanol/propylene glycol (1:1) plus 0.2% by weight Glydant Plus™.

The stratum corneum samples analyzed in this study were taken from male and female subjects (ages 23-54). The studies were conducted as double-blind paired comparisons, with treatment assignments randomized. Subjects first completed a four-day washout phase. During the four-week application phase, test products were applied twice daily to the volar surface of the forearms, using dropper bottles to deliver 0.1 ml of test product to each site; each subject applied a test formulation to one site and the vehicle formulation to the adjacent site. After four weeks of application, samples of stratum corneum were obtained by tape stripping.

A subset of subjects who responded to the treatments as expected, with a significant increase in ceramide levels, data from subjects showing increased ceramides was broken out. In each case, responder subjects represented a cross-section of the whole panel; they were not predominantly of one sex or age group. These subsets of subjects showed significantly increased ceramide levels, with mean increases of 30% and 25% for TAPS alone, and a 38% increase for TAPS+juniperic acid+linoleic acid, compared with vehicle. Increases were seen across all ceramide types. The increases for ceramides 1 and 2 were dramatically higher than for the other ceramide types.

In two separate studies in the UK and Thailand involving 13 and 15 panellists, respectively, an improvement in the scalp condition of dandruff sufferers was shown to correlate with increased ceramide levels of around 300% in the scalp and reduced response to topically applied histamine.

### Example 2

The following is an example of a shampoo composition according to the invention.

| | Example 2 |
|---|---|
| Ingredient | Active ingredient |
| Chemical Name | weight % |
| SLES 2EO | 14 |
| Cocoamidopropylbetaine | 2 |
| Guar | 0.1 |
| hydroxypropyltrimonium | |
| chloride | |
| Dimethiconol | 1 |
| Crosslinked polyacrylic | 0.4 |
| acid | |
| Zinc pyrithione | 0.6 |
| Lactic acid | 0.5 |
| Mica + titanium dioxide | 0.2 |
| Sodium benzoate | 0.5 |
| Water | to 100 |

### Example 3

The following is an example of a rinse-off conditioning composition according to the invention.

| Ingredient | Example 3 |
|---|---|
| Chemical Name | weight % |
| Soft water | q.s. to 100 |
| Citric acid (50% aqueous) | 0.185 |
| Stearamidopropyldimethylamine | 0.5 |
| Propylene glycol | 0.5 |
| Dicetyldimonium chloride/PG | 2.1 |
| Zinc pyrithione | 0.4 |
| Lactic acid | 0.1 |
| TAPS | 0.1 |
| Stearyl alcohol and | 1.0 |
| ceteareth-20 blend | |
| Cetyl alcohol | 3.25 |
| Disodium EDTA | 0.1 |
| Methylchloroisothiazoline/ | 0.05 |
| methylisothiazoline | |
| DMDM hydantoin | 0.1 |
| Potassium hydroxide (50% | 0.033 |
| aqueous) | |
| Fragrance | 0.2 |
| Potassium chloride | 0.1 |

### Example 4

The following shampoo composition according to the invention was prepared:

| | % by weight |
|---|---|
| SLES (2EO) | 16 |
| CAPB | 2 |
| Sunflower Seed Oil | 10 |
| Carbopol TM | 0.4 |
| BHT | 0.024 |
| Water | 71.576 |

| Common | Chemical | Trade | | |
|---|---|---|---|---|
| Name | Name | Name | Function | Supplier |
| SLES (2EO) | Sodium | Empicol | Surfactant | Allbright & |
| | laureth-2 | ESB 3/M | | Wilson |
| | sulphate | | | |
| CAPB | Cocamidopropyl | Tego- | Surfactant | Goldschmidt |
| | betaine | betaine | | |
| Sunflower | Helianthus | N/A | Barrier | Anglia |
| seed oil | Annus | | booster/ | Chemicals |
| | | | emollient | |
| Carbopol | Polyacrylic | Carbopol | Surfactant | Goodrich |
| TM | acid | | | |
| BHT | Butylated | Nipanox | Preservative | Nipa Labs |
| | Hydroxytoluene | BHT | | |

Corneosurfametry (CSM) tests, as described in WO 00/17297, showed that the composition was less damaging to the skin than a corresponding composition containing the same components but with water replacing the sunflower seed oil.

### Example 5

The following is an example of a shampoo composition according to the invention which comprises a solid antimicrobical agent:

| | % by weight |
|---|---|
| SLES (2EO) | 16 |
| CAPB | 2 |
| Sunflower Seed Oil | 10 |
| ZnPTO | 1 |
| Carbopol TM | 0.4 |
| BHT | 0.024 |
| Water | 70.576 |

### Examples 6 and 7

The following are further examples of shampoo compositions according to the invention:

| | Example 6 | Example 7 |
|---|---|---|
| | % by weight | % by weight |
| SLES (2EO) | 12.0 | 12.0 |
| CAPB | 3.0 | 3.0 |
| Cholesterol | 2.5 | 0.5 |
| Sucrose ester | 1.25 | 0.25 |
| Stearic Acid | 1.25 | 0.25 |
| Jaguar C13S* | 0.25 | 0.25 |
| Propylene glycol | 5.00 | 1.00 |
| Formaldehyde | 0.04 | 0.04 |
| Antil 141 TM | 1.5 | 1.5 |
| NaOH to pH 6.0 | | |
| Water to 100% | | |

*Cationic deposition polymer ex Rhodia

### Examples 8 and 9

In vivo studies have shown that shampoos containing sucrose esters (also known as pseudoceramides) are perceived by consumers to give a significant anti-dandruff effect after 2 weeks use as shown in the following table:

| Attribute | Placebo | Example 8 | Example 9 |
|---|---|---|---|
| Makes scalp | 3.8 | 4.7 | 4.8 |
| healthy | | | |
| Gives scalp | 3.5 | 4.3 | 4.6 |
| a fresh | | | |
| feeling | | | |
| No dandruff | 3.5 | 5.3 | 5.3 |
| No | 3.3 | 5.2 | 5.3 |
| itchiness | | | |
| on scalp | | | |

The values were determined using a 7 point scale.

The following compositions were used

| | Example 8* | Example 9** |
|---|---|---|
| | % by weight | % by weight |
| SLES (2EO) | 14.0 | 14.0 |
| CAPB | 2.0 | 2.0 |
| Sucrose ester | 0.025 | 0.025 |
| Jaguar C13S | 0.2 | 0.2 |
| Ethylene glycol distearate | 1.5 | 1.5 |
| Formaldehyde | 0.1 | 0.1 |
| Fragrance | 0.5 | 0.5 |
| Dimethicone | 1.5 | 1.5 |
| NaOH to pH 6.0 | | |
| Water to 100% | | |

*Sucrose ester added to completed shampoo
**Sucrose ester dissolved in perfume

### Example 10

FIG. 1 is a plot showing the relative amount of ceramide in the scalp of a number of subjects. In the Figure, H denoes control subjects having a healthy scalp, SP denotes self perceived dandruff sufferers presenting a healthy scalp and DD denotes self perceived dandruff sufferer presenting clinically recognised symptoms of dandruff. FIG. 1 therefore shows that a depletion in ceramide scalp lipids occurs in dandruff sufferers regardless of whether the sufferers present recognised clinical symptoms of dandruff.

### Example 11

FIG. 3 is a plot showing the relative amount of ceramide in the scalp of a number of subjects. In the Figure, H denotes control subjects having a healthy scalp and I denotes subjects complaining of an itchy scalp. FIG. 3 therefore shows that a depletion in ceramide scalp lipids occurs in people complaining of scalp itch. Furthermore, FIG. 4 is a plot from the same study which shows that people complaining of scalp itch is also accompanied by a reduction in scalp barrier function as measured by corneosurfametry (CSM).

### Example 12

In vitro skin penetration studies demonstrate that topically applied sunflower seed oil triglycerides, in shampoo, penetrate not only into the superficial stratum corneum but also deep into the viable epidermis and dermis, and are therefore available for incorporation into and to enhance the development of the skin barrier.

Radiolabelled formulations were prepared using formulations spiked with either ³H Lysine or ³H glyceryl trioleate in sunflower seed oil. The total balance of each nutrient was brought up to 0.01% by weight using non-labelled cold material. Excised human full thickness skin was obtained and placed in between the two compartments of Franz diffusion cells. The skin is maintained by filling the receptor chamber of the cell with phosphate buffered saline with the cells placed in a water bath at 37° C. to maintain a surface skin tissue of about 32° C. It has been demonstrated that significant amounts of lysine and sunflower seed oil triglyerides (p<0.05) can penetrate not only into the stratum corneum but also deeper into the epidermis and dermis following short contact application. The experimental procedure was as follows:

6-fold diluted shampoo was applied to the skin, left for one minute and washed off. The skin was then separated into 3:

superficial layers of the stratum corneum (this layer was tape stripped and hence identified as “tapes strips”)

remainder of the epidermis (viable) is then removed using heat separation (identified as the epidermal section)

dermis (dermal section).

Each of these layers was analysed for radioactivity and the amount of material penetrated calculated.

Using the in-vitro skin penetration studies, the amount of penetrated material can be quantified as a snap shot in time. From the skin sectioning process, the location of the penetrant at the particular point in time can be measured.

FIG. 2 shows the penetration of triglyceride from shampoo containing sunflower seed oil and zinc pyrithione (ZnPTO) (n=12).

In a further experiment involving an in vitro study using pig skin, it was shown that the triglycerides of sunflower seed oil penetrated the stratum corneum and were converted almost completely to linoleic acid and other fatty acids in two days.

Linoleic and palmitic acid were chosen as representative products from the bioconversion of sunflower seed oil.

The level of ¹⁴C linoleic and palmitic acid used in this experiment was 2.56 μg/cm² skin (mainly linoleic acid).

¹⁴C-labeled palmitic (0.707×10⁻³ mg/ml) and linoleic acids (0.513 mg/ml) (Amersham Corp.) were mixed together at 1:1 (v/v) ratio. 5 μl of this fatty acid mixture was either topically added to human epidermal equivalents (MatTek Corp., Ashland, Mass.; 8 mm diameter), or to the tissue culture media at a final concentration of 1% by weight palmitic/linoleic acid. Untreated epidermal equivalent samples served as controls. Following incubation at 37° C.—5% by volume CO₂ for 48 hours, the human epidermal equivalents were rinsed with 1 ml of sterile phosphate buffered saline to remove excess ¹⁴C-palmitic and linoleic acid before continuing.

Lipid extracts from the samples were prepared as follows: 1 ml of 0.1N NaOH was added to each sample and heated at 70° C. for 1 hour. 150 μl 1N HCl and ⁴⁰⁰ μl 2.5% KCl was then added, mixed, and pH checked to ensure it was below 4 before continuing. 1.5 ml of chloroform:methanol (1:1) was added and mixed for 10 minutes followed by centrifugation for a further 10 minutes at 2000× g. The organic phase was decanted, dried under a stream of nitrogen gas, resuspended in 40 μl of chloroform:methanol (2:1 by volume), and spotted onto a HPTLC plate for subsequent lipid analysis.

The HPTLC plate was first developed in chloroform:methanol:acetic acid (95:4.5:0.5 by volume) to separate the different ceramides. After drying for 10 minutes, the plate was developed again in hexane:ethyl ether:acetic acid (60:40:2 by volume) to further separate the cholesterol and fatty acids. After development, the plate was dried, and saturated with a solution of 10% by weight copper sulphate in 8% by weight phosphoric acid. The plate was heated to 165° C. for 10 minutes to char the lipids for visualization.

The plate was analyzed by autoradiography, and the lipid bands scraped for scintillation counting to quantitate radiolabel incorporation.

Scintillation counting of the lipid bands indicated that the radiolabeled fatty acids indeed have been synthesised into ceramides, demonstrating that human skin in culture can incorporate the major components of sunflower oil into ceramides. The percentages given below represent the molar amount of the radiolabel which was incorporated into the ceramides, when fatty acids were added topically:

| | |
|---|---|
| Ceramide I | 0.14% |
| Ceramide II-V | 0.86% |
| Ceramide VI | 0.25% |

In addition, visual examination of the HPTLC plate also showed that samples treated with fatty acids exhibited larger and more intense lipid bands as compared to the untreated controls.

In summary, this example demonstrates that (1) radiolabeled fatty acids were biosynthesized into ceramides by the human skin equivalents, and (2) fatty acid treated samples had enhanced lipid production compared to the untreated controls.

1. Use of a lipophilic agent or a lipid precursor and/or promoter in the manufacture of a composition for treating and/or preventing dandruff by strengthening the scalp.

2. Use as claimed in claim 1, wherein the composition is a shampoo.

3. Use as claimed in claim 1 or claim 2, wherein the composition comprises an antifungal agent which has activity against Malassezia spp.

4. Use as claimed in claim 3, wherein the antifungal agent is selected from zinc pyrithione, octopirox, climbazole, ketoconazole or mixtures thereof.

5. Use as claimed in claim 3 or claim 4, wherein the antifungal agent is present in the composition in an amount of from 0.01% to 10% by weight.

6. Use as claimed in claim 4 or claim 5, wherein the antifungal agent is zinc pyrithione.

7. Use as claimed in claim 4 or claim 5, wherein the antifungal agent is in solution.

8. Use as claimed in any one of claims 1 to 7, wherein the lipid precursor and/or promoter is present in the composition in an amount of from 0.01% to 20% by weight.

9. Use as claimed in any one of claims 1 to 7, wherein the lipophilic agent is present in the composition in an amount of from 0.001% to 20% by weight.

10. Use as claimed in claim 8, wherein the lipid precursor and/or promoter is present in the composition in an amount of from 0.5% to 10% by weight.

11. Use as claimed in claim 9, wherein the lipophilic agent is present in the composition in an amount of from 0.01% to 10% by weight.

12. Use as claimed in any one of claims 1 to 8 or 10, wherein the lipid precursor and/or promoter is selected from alpha hydroxy acids (AHAs), beta-hydroxy acids (BHAs), omega-hydroxy acids, serine palmitoyl CoA transferase activators, ceramide precursors and mixtures thereof.

13. Use as claimed in any one of claims 1 to 7, 9 or 11, wherein the lipophilic agent is selected from hydrocarbon oils, hydrocarbon waxes, fatty acid derivatives, cholesterol and derivatives thereof, di- and tri- glycerides, vegetable oils and derivatives thereof, liquid non-digestible oils, polyol esters, acetoglyceride esters, alkyl esters and alkenyl esters of fatty acids, lanolin and its derivatives, wax esters, beeswax and its derivatives, sterols, phospholipids, ceramides, neoceramides, pseudoceramides and mixtures thereof.

14. Use as claimed in any one of claims 1 to 7, 9 or 11, wherein the lipophilic agent is a polyol fatty acid polyester.

15. Use as claimed in claim 14, wherein the polyol ester is a sucrose polyester.

16. Use as claimed in claim 14 or 15, wherein the polyol ester comprises two or more C₈ to C₂₂ acyl groups.

17. Use as claimed in claim 16, wherein the C₈ to C₂₂ acyl groups are lauryl groups or are derived from fatty acids obtainable from natural oils such as palm oil, palm kernel oil, soybean oil, coconut oil, fish oil or mixtures thereof.

18. Use as claimed in claim 13, wherein the lipophilic agent is sunflower seed oil.

19. Use of a lipophilic agent or a lipid precursor and/or promoter for strengthening the scalp.

20. Use of a lipophilic agent or a lipid precursor and/or promoter for reducing scalp itch and/or dryness.

21. A method of treating and/or preventing dandruff which comprises strengthening the scalp by applying to the scalp a composition comprising a lipophilic agent or lipid precursor and/or promoter in an amount effective to strengthen the scalp.

22. A method of strengthening the scalp which comprises applying to the scalp a composition comprising a lipophilic agent or lipid precursor and/or promoter in an amount effective to strengthen the scalp.

23. Method as claimed in claim 21 or claim 22 which comprises the steps of :
(a) contacting the scalp with water;
(b) applying to the scalp a lipophilic agent or lipid precursor and/or promoter in an amount effective to penetrate the stratum corneum;
(c) rinsing said lipophilic agent or lipid precursor and/or promoter from the scalp; and
(d) determining the extent to which the dandruff has been treated or the scalp has been strengthened.

24. A method of reducing scalp itch and/or dryness which comprises strenghthening the scalp by a method according to claim 22 or claim 23.
